(19) United States
(12) Patent Application Publication
(10) Pub. No.: US 2005/0008895 A1
(43) Pub. Date: Jan. 13, 2005

(54) Organic electroluminescent material, organic electroluminescent device, and heterocycle-containing iridium complex compound

(76) Inventors: Ichinori Takada, Kanagawa (JP); Tadashi Ishibashi, Kanagawa (JP); Jiro Yamada, Kanagawa (JP); Shinichiro Tamura, Kanagawa (JP)

Correspondence Address: Bell, Boyd & Lloyd LLC, P.O. Box 1135, Chicago, IL, 60690, US

(21) Appl. No.: 10/864,112

(22) Filed: Jun. 8, 2004

(30) Foreign Application Priority Data
Jun. 12, 2003 (JP) P2003-167828

(51) Int. Cl. H05B 3/14; C09K 11/06; C07D 235/04

(52) U.S. Cl. 428/690; 428/917; 313/504; 313/506; 548/103; 548/108; 252/301.16

(57) ABSTRACT

A heterocycle-containing iridium complex compound which emits light in the region from green to blue, and an organic EL device using the iridium complex providing higher efficiency and extended lifespan are provided. The EL device includes, for example, an organic layer including a heterocycle-containing iridium complex compound represented by the following structural formula:

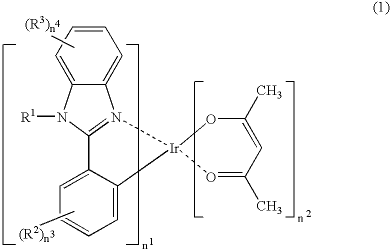

### CROSS REFERENCES TO RELATED APPLICATIONS

This application claims priority to Japanese Patent Application No. P2003-167828 filed on Jun. 12, 2003, the disclosure of which is incorporated by reference herein.

### BACKGROUND OF THE INVENTION

The present invention generally relates to an organic electroluminescent material and an organic electroluminescent device employing same. More specifically, present invention relates to an organic electroluminescent material, an organic electroluminescent device, and a heterocycle-containing iridium complex compound having light emission properties in the region from green to blue, which is advantageously used as an organic electroluminescent material.

An organic electroluminescent (organic EL) display has advantages in that the colors are clear and the panel can be manufactured so as to have reduced thickness, and has as a consequence received attention as a candidate for the next-generation flat panel display. However, in order to bring it into practical use, there are inevitable issues of improving materials to increase the light emission efficiency and prolonging the lifetime of light emission. In recent years, in order to solve the problem, the development of phosphorescent materials has been vigorously conducted, but materials emitting light in the region from green to blue are still in a developing stage.

Generally, in the excitation of a light-emitting material, a process in which the material is deactivated after emitting a fluorescent light and a process in which the material undergoes intersystem crossing and then is deactivated as a phosphor are known, and it is considered that a high theoretical efficiency can be obtained in the latter process. Recently, it has been reported that a complex of a transition metal (especially iridium) has large intersystem crossing and can exhibit high EL emission efficiency. See, Thompson et al.: J. Am. Chem. Soc. 2001, 123, 4305.

However, in the development of a material which emits light in the region from green to blue, studies must be made on ligand. An attempt to substitute a ligand with an electron attractive substituent, such as a fluorine atom, is currently being made, for example, according to Thompson et al.: ICEL. 2001, p.45, but it is important to combine an electron-donating substituent and an electron attractive substituent to balance the electron attractive property with the electron-donating property.

### SUMMARY OF THE INVENTION

The present invention generally relates to an organic electroluminescent material and an organic electroluminescent device employing same. More specifically, present invention relates to an organic electroluminescent material, an organic electroluminescent device, and a heterocycle-containing iridium complex compound having light emission properties in the region from green to blue, which is advantageously used as an organic electroluminescent material.

The present invention provides a iridium complex compound which emits light in the region from green to blue, and provides an organic EL device using the iridium complex compound, thus providing higher efficiency and extended lifespan.

The inventors of the present invention have conducted intensive studies with a view toward developing a iridium complex material having phosphorescence in the region from green to blue. As a result, they have found that, by introducing a number of nitrogen atoms into the heterocycle in the molecule of a ligand and substituting the two benzene rings in the ligand with an electron attractive substituent and an electron-donating substituent to control the electronic state of the ligand, a heterocycle-containing iridium complex compound having excellent light emission properties can be obtained pursuant to an embodiment of the present invention.

In an embodiment, the present invention provides an organic electroluminescent material including a heterocycle-containing iridium complex compound represented by the following general formula (1):

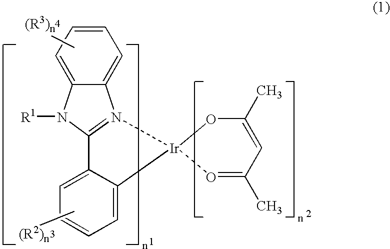

In the formula, R¹ represents any one of a lower alkyl group, a phenyl group, a substituted phenyl group and the like, R² and R³ are the same or different and include, for example, an alkyl group, an alkyloxy group, a cyano group and the like, each of n¹ and n² represents an integer, such as the combinations [n¹=2, n²=1] and [n¹=3, n²=0], and each of n³ and n⁴ independently represents an integer, such as from 0 to 4.

In another embodiment, the present invention provides an organic electroluminescent device that includes an organic layer having a number of layers and having therebetween a pair of electrodes including at least a light emission region, wherein the organic electroluminescent device includes at least one layer of the organic layer that includes at least one heterocycle-containing iridium complex compound represented by the following general formula (1): 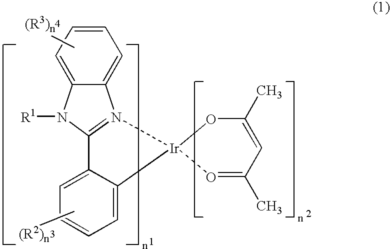
wherein R¹ represents any one of a lower alkyl group, a phenyl group, a substituted phenyl group and the like, R² and R³ are the same or different, such as an alkyl group, an alkyloxy group, a cyano group and the like, each of n¹ and n² represents an integer, such as the combinations [n¹=2, n²=1] and [n¹=3, n²=0], and each of n³ and n⁴ independently represents an integer, such as from 0 to 4.

In yet another embodiment, the present invention provides a heterocycle-containing iridium complex compound represented by the following general formula 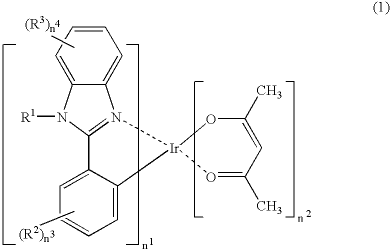
wherein R¹ represents any one of a lower alkyl group, a phenyl group, a substituted phenyl group and the like, R² and R³ are the same or different, such as an alkyl group, an alkyloxy group, a cyano group and the like, each of n¹ and n² represents an integer, such as the combinations [n¹=2, n²=1] and [n¹=3, n²=0], and each of n³ and n⁴ independently represents an integer, such as 0 to 4.

The present invention in an embodiment provides a novel and unique heterocycle-containing iridium complex compound represented by the general formula (1) described above. The heterocycle-containing iridium complex compound can be advantageously used as an organic electroluminescent material having excellent light emission properties in the region from green to blue.

Further, by forming an organic layer containing a light emission region using an organic electroluminescent material that includes the heterocycle-containing iridium complex compound according to a preferred embodiment of the present invention, there can be provided an EL device which emits light in the region from green to blue with high efficiency and extended lifespan.

Additional features and advantages of the present invention are described in, and will be apparent from, the following Detailed Description of the Invention and the figures.

### BRIEF DESCRIPTION OF THE FIGURES

FIG. 1 shows a diagrammatic cross-sectional view of one example of the structure of a bottom emission type organic EL device according to a preferred embodiment of the present invention.

FIG. 2 shows diagrammatic cross-sectional view showing one example of the structure of a top emission type organic EL device according to a preferred embodiment of the present invention.

FIG. 3 shows a diagrammatic cross-sectional view showing the basic structure of a top emission type organic EL device according to a preferred embodiment of the present invention.

### DETAILED DESCRIPTION OF THE INVENTION

The present invention generally relates to an organic electroluminescent material and an organic electroluminescent device employing same. More specifically, present invention relates to an organic electroluminescent material, an organic electroluminescent device, and a heterocycle-containing iridium complex compound having light emission properties in the region from green to blue, which is advantageously used as an organic electroluminescent material.

The heterocycle-containing iridium complex compound represented by the general formula (1) described above used in an embodiment of the present invention includes a novel and unique compound. In a preferred embodiment, the heterocycle-containing iridium complex compound is represented by any one of the following structural formulae (2-1) to (2-4).

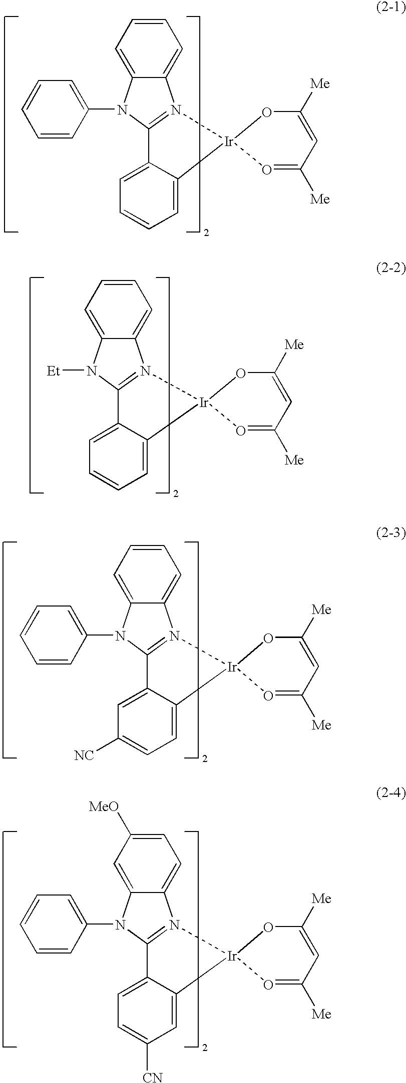

The heterocycle-containing iridium complex compound according to a preferred embodiment of the present invention can be obtained by, for example, by a preparation method in which monomers corresponding to the degree of polymerization n¹ in the general formula (1) described above are reacted in sodium hexachloroiridate and 2-methoxyethanol (Nonoyama's method (Bull. Chem. Soc. Jpn. 1794, 47, 767.)), and then heated in 2-ethoxyethanol, together with 1,4-pentanedione and sodium carbonate, followed by purification (Lamansky et al. method (Inorg. Chem. 2001, 40, 1704.)).

The heterocycle-containing iridium complex compound according to a preferred embodiment of the present invention can be advantageously used as an organic electroluminescent material which emits light in the region from green to blue, and, using an organic electroluminescent material comprising the iridium complex compound, an organic electroluminescent device having high light emission efficiency and extended lifespan can be provided.

The heterocycle-containing iridium complex compound according to a preferred embodiment of the present invention can be used not only as a material for the above-mentioned organic electroluminescent device but also in medical applications, a fluorescent brightener, a material for photography, a UV absorbing material, a laser dye, a dye for color filter, a color conversion filter and suitable other applications.

An organic electroluminescent device according to a preferred embodiment of the present invention will be described below in greater detail with reference to the drawing.

FIG. 3 shows a diagrammatic cross-sectional view of an example of the organic electroluminescent device according to a preferred embodiment of the present invention. This organic electroluminescent device includes an anode 12, an organic layer 13 containing a light emission region, and a cathode 14 formed in this order on a substrate 11. This organic electroluminescent device is a top emission type organic electroluminescent device which emits light from the cathode side.

The organic electroluminescent device may have, between the electrodes, an organic layer, such as a light emitting layer, a hole injection layer, a hole transport layer, a hole blocking layer, or an electron transport layer, and a protecting layer, and the like, and, by appropriately selecting the material, a layer other than the light emitting layer in these layers or the interface between the layers may emit light.

In the substrate 11, glass, a plastic, or another appropriate material can be used, but preferred is glass which most advantageously suppresses penetration of moisture from the outside. When the organic electroluminescent device and another display device are used in combination, the substrate 11 can be common to them. In the anode 12, a stacked structure including a highly reflective metal material, such as chromium (Cr), or an ITO layer and an Ag alloy can be used.

The organic layer 13 contains an organic electroluminescent material including the heterocycle-containing iridium complex compound represented by the general formula (1) described above.

The heterocycle-containing iridium complex compound according to a preferred embodiment of the present invention as an organic electroluminescent material has properties such that it emits blue light with high efficiency, and therefore, when the compound is contained in the light emitting layer in the organic layer, the electroluminescent device can be improved in light emission efficiency. In addition, the heterocycle-containing iridium complex compound has excellent charge transportability, and therefore, when the compound is contained in the charge transport layer, the electroluminescent device can be improved in light emission efficiency. Therefore, there can be provided an electroluminescent device which is advantageous not only in that it can reduce energy consumption during the light emission, but also in that it can continue blue light emission with high luminance for a long time.

In the electroluminescent device as mentioned above, it is preferred that a layer containing a compound having an ionization potential of about 5.9 eV or higher, more preferably about 6.0 to about 7.0 eV, is disposed between the cathode and the light emitting layer, and it is more preferred that an electron transport layer having an ionization potential of about 5.9 eV or higher is disposed. Any suitable and appropriate materials can be used in the individual layers. In such electroluminescent device, the heterocycle-containing iridium complex compound can be contained not only in the light emitting layer in the organic layer as a light emitting material, but also in the charge transport layer in the organic layer.

With respect to the method for forming a layer containing the heterocycle-containing iridium complex compound, there is no particular limitation, and various methods, such as a vacuum deposition method, an LB method, a resistive heating deposition method, an electron beam method, a sputtering method, a molecule stacking method, a coating method (e.g., a spin coating method, a casting method, or a dip coating method), an ink-jet method, a printing method and the like, can be employed, and, from the viewpoint of obtaining excellent properties and facilitating the preparation, a resistive heating deposition method is preferred.

As an electrode material for the cathode 14, an alloy of an active metal, such as Li, Mg, Ca or the like, and a metal, such as Ag, Al, In or the like, or a stacked structure including these metals can be used. In the organic electroluminescent device, the thickness of the cathode is controlled to obtain a thickness such that the device transmits light emitted from the cathode side.

In the above embodiment, an explanation is made on the so-called top emission type organic electroluminescent device which emits light from the cathode side on top, but the organic electroluminescent device according to a preferred embodiment of the present invention is not limited to this type and can also be applied to a so-called bottom emission type organic electroluminescent device which emits light from the anode side on bottom. The bottom emission type organic electroluminescent device includes, for example, a light transmitting anode comprised of ITO or the like formed on a glass substrate, a light emitting layer formed on the anode, and a light reflecting cathode formed on the light emitting layer. The organic electroluminescent device can be applied to a transmission type organic electroluminescent device including an anode and a cathode both of which are comprised of a light transmitting material, such as ITO.

The organic electroluminescent device according to a preferred embodiment of the present invention can be used in applications in a wide variety of fields, such as display device, display, backlight, electrophotography, illuminating light source, recording light source, exposure light source, reading light source, sign, advertising board, interior, optical communication and the like.

Examples of preferred embodiments of the present invention are provided below without limitation to the scope of the present invention.

### EXAMPLE 1

A heterocycle-containing iridium complex compound represented by the structural formula (2-1) described above was synthesized in accordance with the following reaction formula.

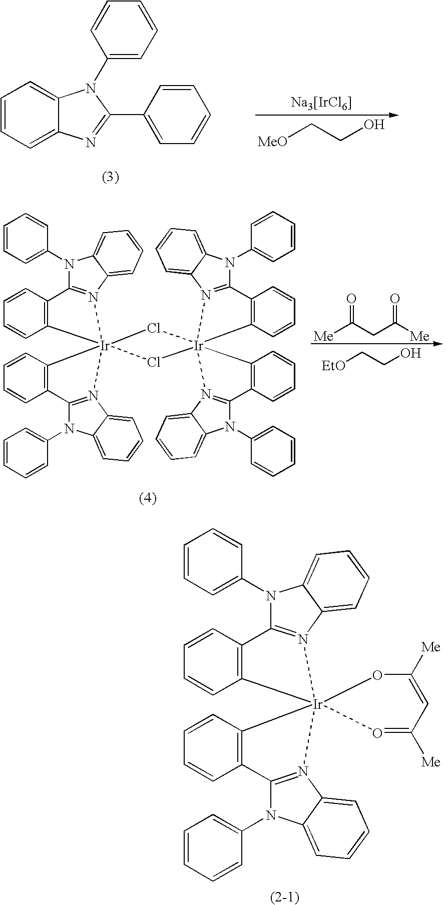

The compound represented by the structural formula 4 in the above-described reaction formula was synthesized from the compound represented by the structural formula 3 in the same reaction formula in accordance with the Nonoyama's method (See, Bull. Chem. Soc. Jpn. 1794, 47, 767.). The heterocycle-containing iridium complex compound represented by the structural formula (2-1) above was synthesized from the compound of the structural formula 4 above in accordance with the method of Lamansky et al. (See, Inorg. Chem. 2001, 40, 1704.).

Specifically, the compound of the structural formula 3 (2.7 g, 9.9 mmol) and sodium hexachloroiridate (1.6 g, 3.3 mmol) were stirred in 2-methoxyethanol for 24 hours. The resultant precipitate (compound of the structural formula 4) was washed with ethanol again, and then heated under reflux in 2-ethoxyethanol for 15 hours, together with 1,4-pentanedione (6.7 g, 66 mmol) and sodium carbonate (1.4 g, 13 mmol). The resultant insoluble substance was collected by filtration, and washed with water, ethanol, ether, and hexane. The residue was purified by silica gel chromatography (developing solvent: methylene chloride), and permitted to undergo sublimation to obtain the compound of the structural formula (2-1) (410 mg; yield based on sodium hexachloroiridate: 15%).

Data for identification of the compound of the structural formula (2-1) above are provided as follows.

(1) ¹H NMR (CD₂Cl₂/ppm); δ 1.9 (s, 6H), 5.3 (s, 1H), 6.4-6.6 (8H), 7.1-7.2 (2H), 7.2-7.3 (4H), 7.6-7.7 (10H), 7.7-7.8 (2H);

(2) MS (FAB) [M]⁺: 829;

(3) Abs (CH₂Cl₂) 410 nm; and

(4) PL (CH₂Cl₂) 520 nm.

### EXAMPLE 2

A heterocycle-containing iridium complex compound represented by the structural formula (2-2) described above was synthesized in accordance with substantially the same procedure as in Example 1 except that, instead of the compound of the structural formula 3 in Example 1, a compound of the structural formula discussed below was used. As a result, the yield (based on sodium hexachloroiridate) of the iridium complex compound was found to be 12%.

Chemical formula of Example 2

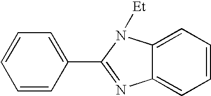

Data for identification of the above iridium complex compound are as follows.

(1) ¹H NMR (CD₂Cl₂/ppm); δ 1.3 (t, 6H), 1.9 (s, 6H), 2.3 (q, 4H), 5.2 (s, 1H), 6.4-6.5 (4H), 7.1-7.2 (2H), 7.3-7.6 (8H), 7.7-7.8 (2H);

(2) MS (FAB) [M]⁺: 733;

(3) Abs (CH₂Cl₂) 410 nm; and

(4) PL (CH₂Cl₂) 520 nm.

### EXAMPLE 3

A heterocycle-containing iridium complex compound represented by the structural formula (2-3) above was synthesized in accordance with substantially the same procedure as in Example 1 except that, instead of the compound of the structural formula 3 in Example 1, a compound of the structural formula below was used (CAS No. 175712-80-8). As a result, the yield (based on sodium hexachloroiridate) of the iridium complex compound was found to be 15%.

Chemical formula of example 3

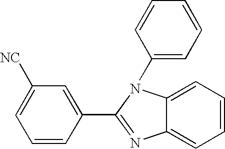

Data for identification of the above iridium complex compound are as follows.

(1) ¹H NMR (CD₂Cl₂/ppm); δ 1.8 (s, 6H), 5.3 (s, 1H), 6.4-7.2 (8H), 7.2-7.3 (4H), 7.5-7.6 (10H), 7.7-8.0 (4H);

(2) MS (FAB) [M]⁺: 879;

(3) Abs (CH₂Cl₂) 410 nm; and

(4) PL (CH₂Cl₂) 500 nm.

### EXAMPLE 4

A compound represented by the structural formula 6 below was synthesized from a compound represented by the structural formula 5 below in accordance with the following reaction formula:

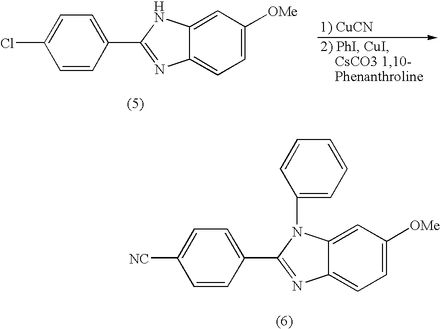

Specifically, the compound of the structural formula 5 above (synthesized in accordance with Eur. J. Med. Chem. 1996, 31, 635.)(5.0 g, 19 mmol) and copper(I) cyanide (8.7 g, 98 mmol) were heated under reflux in 500 ml of dioxane for 12 hours. The reaction solution was concentrated, washed with water, and dried, and to the resultant solid were added iodobenzene (3.9 g, 19 mmol), cesium carbonate (19 g, 58 mmol), copper(I) iodide (370 mg, 1.9 mmol), 1,10-phenanthroline (700 mg, 3.9 mmol), and 200 ml of dioxane, followed by heating under reflux for 36 hours. The resultant reaction mixture was concentrated, and purified by silica gel chromatography (developing solvent: hexane-chloroform-toluene) to obtain 1.5 g of the compound represented by the structural formula 6 above. Complete purification was difficult, and therefore clear spectral data was not obtained, but, in the measurement of MS spectrum, a molecular ion peak ([M]⁺: 326) was observed.

Then, using the compound represented by the structural formula 6, a heterocycle-containing iridium complex compound represented by the structural formula (2-4) above was synthesized. The procedure for the synthesis of this compound is the same as that for the heterocycle-containing iridium complex compound represented by the structural formula (2-1) above.

Data for identification of the above heterocycle-containing iridium complex compound are provided as follows:

(1) ¹H NMR (CD₂Cl₂/ppm); δ 1.8 (s, 6H), 3.5 (s, 6H), 5.3 (s, 1H), 6.4-7.2 (6H), 7.2-7.3 (4H), 7.6-7.7 (10H), 7.7-8.0 (4H);

(2) MS (FAB) [M]⁺: 889;

(3) Abs (CH₂Cl₂) 400 nm; and

(4) PL (CH₂Cl₂) 480 nm.

### EXAMPLE 5

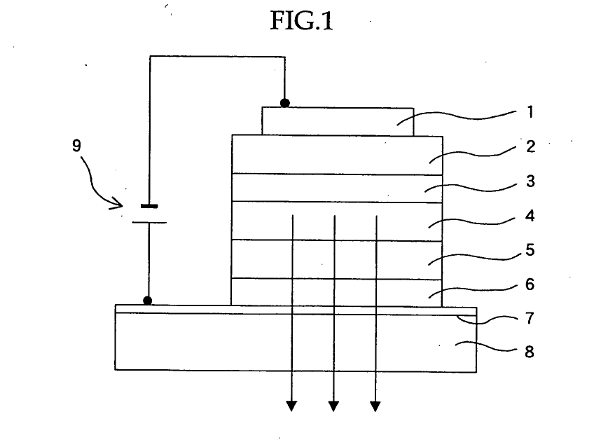

The present example is an example in which an organic electroluminescent device was prepared using the heterocycle-containing iridium complex compound represented by the structural formula (2-1) described above prepared in Example 1 as a light emitting material. The structure of this electroluminescent device is diagrammatically shown in FIG. 1, and the reference numerals are as follows: 1: cathode; 2: electron transport layer; 3: hole blocking layer; 4: light emitting layer; 5: hole transport layer; 6: hole injection layer; 7: anode; 8: substrate; and 9: power source.

First, a 30 mm×30 mm glass substrate having formed on one surface an anode that includes ITO having a thickness of 100 nm was set in a vacuum deposition machine. As a deposition mask, a metal mask having a plurality of 2.0 mm×2.0 mm unit apertures was disposed near the substrate, and CuPc (copper phthalocyanine) was deposited as a hole injection layer by a vacuum deposition method in a vacuum at 10⁻⁴ Pa or less so that the thickness of the resultant layer became 10 nm. The deposition rate was 0.1 nm/second.

Then, α-NPD represented by the structural formula below was deposited as a hole transport layer material directly on the hole injection layer. The thickness of the hole transport layer comprised of α-NPD was 30 nm, and the deposition rate was 0.1 nm/second.

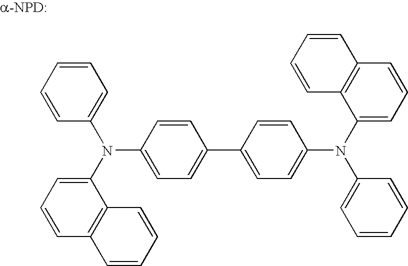

Subsequently, a light emitting layer that includes the iridium complex represented by the structural formula (2-1) above and CBP (carbazolebiphenyl), which were mixed in a 94:6 weight ratio, was deposited directly on the hole transport layer. The thickness of the light emitting layer was 40 nm.

Then, BCP (bathocuproine) was deposited as a hole blocking layer material directly on the light emitting layer. The thickness of the hole blocking layer that includes BCP was 10 nm, and the deposition rate was 0.1 nm/second.

Alq₃ {tris(8-hydroxyquinolinato)aluminum} represented by the structural formula below was deposited as an electron transport layer material directly on the hole blocking layer. The thickness of the electron transport layer comprised of Alq₃ was 30 nm, and the deposition rate was 0.2 nm/second.

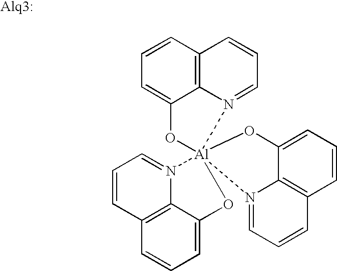

As materials for the cathode, a Mg and Ag co-deposited film was used and deposited at a deposition rate of 1 nm/second so that the thickness of the resultant film became 200 nm, thus preparing an organic electroluminescent device having the stacked structure shown in FIG. 1.

A forward bias direct voltage was applied to the thus prepared organic electroluminescent device in Example 5 in a nitrogen gas atmosphere to evaluate the light emission properties. The light emitted was green, and a spectrophotometry measurement offered a spectrum having an emission peak around 505 nm. In the spectrophotometry measurement, a spectrophotometer using a photodiode array, manufactured and sold by Otsuka Electronics Co., Ltd., as a detector was used. In addition, a voltage-luminance measurement was carried out, and, as a result, a luminance of 800 cd/m² was obtained at 8 V.

The organic electroluminescent device prepared was allowed to stand in a nitrogen gas atmosphere for one month, but no deterioration was observed in the device. Further, a fixed current was permitted to flow through the organic electroluminescent device at an initial luminance of 500 cd/m² so that the device continuously emitted light and suffered forced deterioration. As a result, it was found that a 900-hour period of time was required until the luminance reduced by half. The results are summarized in the Table 1 below.

### EXAMPLE 6

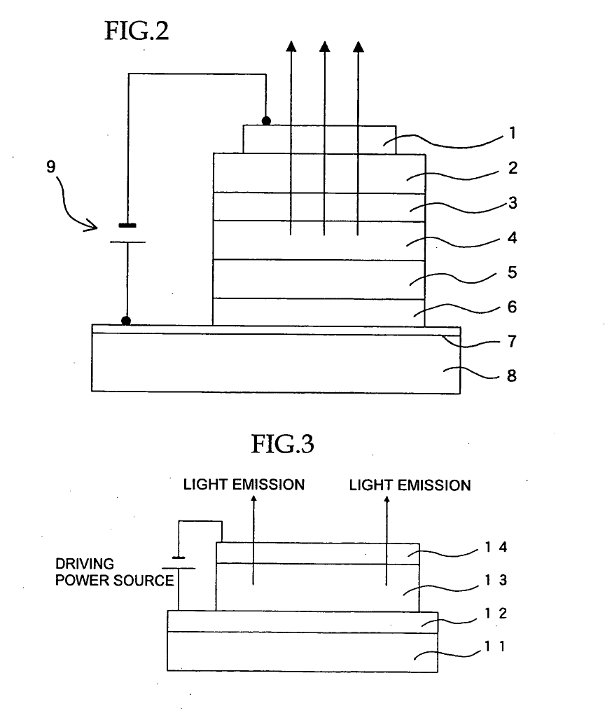

The present Example is an example in which a top emission type organic electroluminescent device was prepared using the iridium complex compound represented by the structural formula (2-1) above as a light emitting material. The structure of this electroluminescent device is diagrammatically shown in FIG. 2, and like parts or portions are indicated by like reference numerals in FIG. 1 and FIG. 2.

As a substrate, using a 30 mm×30 mm glass substrate having formed on one surface an anode that includes Cr having a thickness of 100 nm, an organic electroluminescent device having the stacked structure shown in FIG. 2 was prepared. The other procedure for preparing the device and the materials are substantially the same as those in Example 1, except for the device structure. As a constituent material for the cathode 1, a Mg/Ag co-deposited film was used. The thickness of the co-deposited film was 11 nm.

A forward bias direct voltage was applied to the thus prepared organic electroluminescent device in Example 6 in a nitrogen gas atmosphere to evaluate the light emission properties. The light emitted was bluish green, and a spectrophotometry measurement offered a spectrum having an emission peak around 505 nm. In the spectrophotometry measurement, a spectrophotometer using a photodiode array, manufactured and sold by Otsuka Electronics Co., Ltd., as a detector was used. In addition, a voltage-luminance measurement was carried out, and, as a result, a luminance of 570 cd/m² was obtained at 8 V.

The organic electroluminescent device prepared was allowed to stand in a nitrogen gas atmosphere for one month, but no deterioration was observed in the device. Further, a fixed current was permitted to flow through the organic electroluminescent device at an initial luminance of 500 cd/m² so that the device continuously emitted light and suffered forced deterioration. As a result, it was found that a 780-hour period of time was required until the luminance reduced by half. The results are summarized in the Table 1 below.

### EXAMPLE 7

The present Example is an example in which an organic electroluminescent device having the structure shown in FIG. 1 was prepared using the iridium complex compound represented by the structural formula (2-2) above as a light emitting material. The procedure for preparing the device, the device structure, and the materials are substantially the same as those in Example 5, except for the light emitting material. The results are summarized in the Table 1 below.

### EXAMPLE 8

The present Example is an example in which an organic electroluminescent device having the structure shown in FIG. 2 was prepared using the iridium complex compound represented by the structural formula (2-2) above as a light emitting material. The procedure for preparing the device, the device structure, and the materials are substantially the same as those in Example 6, except for the light emitting material. The results are summarized in the Table 1 below.

### EXAMPLE 9

The present Example is an example in which an organic electroluminescent device having the structure shown in FIG. 1 was prepared using the iridium complex compound represented by the structural formula (2-3) above as a light emitting material. The procedure for preparing the device, the device structure, and the materials are substantially the same as those in Example 5, except for the light emitting material. The results are summarized in the Table 1 below.

### EXAMPLE 10

The present Example is an example in which an organic electroluminescent device having the structure shown in FIG. 2 was prepared using the iridium complex compound represented by the structural formula (2-3) above as a light emitting material. The procedure for preparing the device, the device structure, and the materials are substantially the same as those in Example 6, except for the light emitting material. The results are summarized in the Table 1 below.

### EXAMPLE 11

The present Example is an example in which an organic electroluminescent device having the structure shown in FIG. 1 was prepared using the iridium complex compound represented by the structural formula (2-4) above as a light emitting material. The procedure for preparing the device, the device structure, and the materials are substantially the same as those in Example 5, except for the light emitting material. The results are summarized in the Table 1 below.

### EXAMPLE 12

The present Example is an example in which an organic electroluminescent device having the structure shown in FIG. 2 was prepared using the iridium complex compound represented by the structural formula (2-4) above as a light emitting material. The procedure for preparing the device, the device structure, and the materials are substantially the same as those in Example 6, except for the light emitting material. The results are summarized in the Table 1 below.

TABLE 1

| | | | Color of | Voltage- | |
|---|---|---|---|---|---|
| Exam- | | Device | emitted | luminance | |
| ple | Compound | structure | light | property | Lifespan |
| 1 | Structural | | 505 nm | 800 cd/m² | 900 hr |
| | formula (2-1) | | (Green) | (8 V) | (500 cd/m²) |
| 2 | | | 505 nm | 570 cd/m² | 780 hr |
| | | | (Green) | (8 V) | (500 cd/m²) |
| 3 | Structural | | 505 nm | 780 cd/m² | 910 hr |
| | formula (2-2) | | (Green) | (8 V) | (500 cd/m²) |
| 4 | | | 505 nm | 580 cd/m² | 800 hr |
| | | | (Green) | (8 V) | (500 cd/m²) |
| 5 | Structural | | 500 nm | 850 cd/m² | 830 hr |
| | formula (2-3) | | (Green) | (8 V) | (500 cd/m²) |
| 6 | | | 500 nm | 595 cd/m² | 710 hr |
| | | | (Green) | (8 V) | (500 cd/m²) |
| 7 | Structural | | 480 nm | 900 cd/m² | 600 hr |
| | formula (2-4) | | (Green) | (8 V) | (500 cd/m²) |
| 8 | | | 480 nm | 610 cd/m² | 540 hr |
| | | | (Green) | (8 V) | (500 cd/m²) |

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1. An organic electroluminescent material comprising a heterocycle-containing an iridium complex compound represented by the following formula (1): 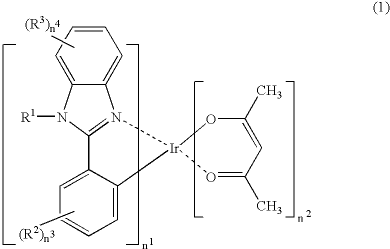
where
R¹ is a lower alkyl group, a phenyl group, or a substituted phenyl group;
R² and R³ are selected from the group consisting of an alkyl group, an alkyloxy group, and a cyano group;
each of n¹ and n² represents an integer number selected from the group consisting of n¹=2, n²=1 and n¹=3, n²=0; and
each of n³ and n⁴ independently represents an integer ranging from 0 to 4.

2. An organic electroluminescent device comprising an organic layer having a plurality of layers and having between a pair of electrodes at least a light emission region, wherein at least one of the layers of the organic layer include at least one heterocycle-containing iridium complex compound represented by the following formula (1): 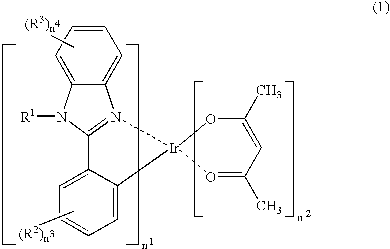
where R¹ represents a lower alkyl group, a phenyl group, or a substituted phenyl group;
R² and R³ are selected from the group consisting of an alkyl group, an alkyloxy group, and a cyano group;
each of n¹ and n² represents an integer selected from the group consisting of n¹=2, n²=1 and n¹=3, n²=0; and
each of n³ and n⁴ independently represents an integer ranging from 0 to 4.

3. A composition comprising a heterocycle-containing iridium complex compound represented by the following formula (1): 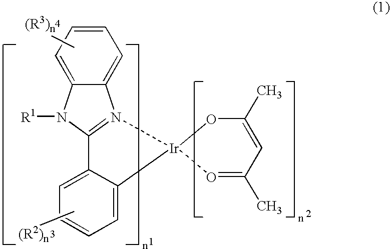
where R¹ represents a lower alkyl group, a phenyl group, or a substituted phenyl group;
R² and R³ are selected from the group consisting of an alkyl group, an alkyloxy group, and a cyano group;
each of n¹ and n² represents an integer selected from the group consisting of n¹=2,n²=1 and n¹=3, n²=0; and
each of n³ and n⁴ independently represents an integer ranging from 0 to 4.

4. The composition according to claim 3, the heterocycle-containing iridium complex compound is further represented by any one of the following structural formulae (2-1) to (2-4): [chemical structure]
